(19) United States
(12) Patent Application Publication
(10) Pub. No.: US 2008/0058927 A1
(43) Pub. Date: Mar. 6, 2008

(54) Ossicular Prostheses Fabricated From Shape Memory Polymers

(76) Inventor: Robert Brosnahan, Germantown, TN (US)

Correspondence Address: GORDON & JACOBSON, P.C., 60 LONG RIDGE ROAD, SUITE 407, STAMFORD, CT, 06902, US

(21) Appl. No.: 11/845,385

(22) Filed: Aug. 27, 2007

Related U.S. Application Data
(60) Provisional application No. 60/823,914, filed on Aug. 30, 2006.

(51) Int. Cl. A61F 2/18

(52) U.S. Cl. 623/10

(57) ABSTRACT

Ossicular replacement prostheses are manufactured from a non-biodegradable shape memory polymer. Such prostheses can include TORPs, PORPs, and incudo-stapedial joints (ISJs). The prostheses are reshaped upon application of a stimulus to capture a portion of one or more ossicles. The force of capture of a reshaped polymeric prosthesis is less than a comparable reshaped shape memory alloy prosthesis and thereby prevents recipient discomfort and/or pressure induced necrosis of the bone. In addition, biocompatible shape memory polymers can be designed with recoverable strain that is orders of magnitude higher than shape memory alloys. Furthermore, prostheses can be manufactured in a single size and easily trimmed or otherwise modified by the surgeon during the implantation procedure to tailor the prosthesis in size and/or shape for the particular anatomy. Such modification does not negatively effect the ability of the prostheses to engage the ossicle.

### CROSS-REFERENCE TO RELATED APPLICATIONS

This application claims the benefit of U.S. Provisional No. 60/823,914, filed Aug. 30, 2006, which is hereby incorporated by reference herein in its entirety.

### BACKGROUND OF THE INVENTION

1. Field of the Invention

This invention relates broadly to implantable prostheses. More particularly, this invention relates to prostheses for total or partial replacement of the ossicular bones or joints of such bones.

2. State of the Art

Hearing is facilitated by the tympanic membrane transforming sound in the form of acoustic sound waves within the outer ear into mechanical vibrations within the ossicular chain of bones in the middle ear. The mechanical vibrations are transmitted to the oval window where pressure on the oval window membrane causes compression waves within the fluid of the inner ear. The compression waves lead to vibrations of the cilia (hair cells) located within the cochlear where they are translated into nerve impulses. The nerve impulses are sent to the brain via the cochlear nerve and are interpreted in the brain as sound.

Due to disease, trauma, or congenital malformation, the ossicles of the middle ear are sometimes damaged. Hearing efficiency can be lost to erosion of the ossicular bones: maleus, incus, and stapes. These bones can be completely replaced by a prosthesis (total ossicular replacement prosthesis, or TORP) or replaced in part (partial ossicular replacement prosthesis, or PORP).

In addition, the delicate joint between the incus and the stapes is termed the incudo-stapedial joint (ISJ). The ISJ is a cartilaginous joint having a tendency to ossify in older humans. When the joint is interrupted due to erosion of the joint or the incus itself, vibrations can no longer be transmitted from the incus to the stapes. The result is a conductive hearing loss related to the disrupted ossicular chain.

### SUMMARY OF THE INVENTION

Ossicular replacement prostheses are manufactured from shape memory polymers. Prostheses are provided for TORPS, PORPS, including but not limited to pistons, as well as the incudo-stapedial joint (ISJ). The shape memory polymers can be either thermoplastic or thermoelastic, with low toxicity. In various embodiments, the prostheses are reshaped upon application of a stimulus to capture a portion of one or more ossicles. The force of capture of a reshaped polymeric prosthesis is less than a comparable reshaped shape memory alloy prosthesis and thereby prevents recipient discomfort and/or pressure induced necrosis of the bone. In addition, the shape memory material has relative low mass which means it is can be more easily acted upon. It also has no nickel which could otherwise cause a negative reaction in sensitive individuals. Moreover, it is recognized that the shape memory polymer it relatively MRI inactive, in distinction from metals, which are often used in ossicular prostheses.

Furthermore, the prostheses for replacement of the various ossicles and joints can be manufactured in a single size and easily trimmed or otherwise modified by the surgeon during the implantation procedure to tailor the prosthesis in size and/or shape for the particular anatomy. Such modification does not negatively effect, and may improve, the ability of the prostheses to engage a portion of the ossicle. Moreover, the ability to tailor size and shape permits a limited inventory of prostheses.

Additional objects and advantages of the invention will become apparent to those skilled in the art upon reference to the detailed description taken in conjunction with the provided figures.

### BRIEF DESCRIPTION OF THE DRAWINGS

FIGS. 1 and 2 are side elevation views of a total ossicular replacement prosthesis (TORP) made from shape memory polymer according to the invention, shown at a first length and stimulated second length;

FIG. 3 is a side elevation view of another embodiment of a TORP made from a shape memory polymer according to the invention;

FIG. 4 is a side elevation view of a stapedial prosthesis, more specifically a piston, made from a shape memory polymer according to the invention;

FIG. 5 shows the stapedial prosthesis of FIG. 4 with a central opening mechanically enlarged after molding and heat setting;

FIG. 6 shows the stapedial prosthesis of FIGS. 4 and 5 after application of stimulus to cause shape change;

FIG. 7 is a side elevation view of a incudo-stapedial joint (ISJ) prosthesis made from a shape memory polymer according to the invention;

FIG. 8 is a top view of the ISJ prosthesis of FIG. 7;

FIG. 9 is a section view across line 9-9 in FIG. 8; and

FIG. 10 is a bottom view of the ISJ prosthesis of FIG. 7.

### DETAILED DESCRIPTION OF THE PREFERRED EMBODIMENTS

In accord with the invention, an otological prosthesis such as a total or partial ossicular replacement prosthesis (TORP or PORP) or an incudo-stapedial joint (ISJ) prosthesis is manufactured from a shape memory polymer. Shape memory polymers may be thermoelastic or thermoplastic. For otological prostheses it is important that the shape memory polymer, while being biocompatible also be non-biodegradable and thus suitable for long-term implantation. One suitable class of polymers includes acrylics of methacrylate. More particularly, one preferred shape memory polymer suitable for biomedical applications, and specifically for fabrication of an ossicular prosthesis, is synthesized by photopolymerization from a tert-Butyl acrylate monomer with a diethyleneglycol-dimethacrylate crosslinker. A shape memory polymer as discussed above is available from MedShape Solutions, Inc. of Atlanta, Ga. It is appreciated that other shape memory polymers, such as Calo.MER™ shape memory thermoplastic available from The Polymer Technology Group of Berkeley, Calif. also may be suitable. Other biocompatible shape memory polymers suitable for long term implantation can also be used.

A preferred shape memory polymer has a density of approximately 0.043 lbs/in³, approximately one-fifth the density of the nickel titanium shape memory alloy, Nitinol, (0.235 lbs/in³), which is another material used in ossicular prostheses. The significantly less dense shape memory polymer material results in an implant of reduced mass. Such implant is easier for the remaining otological structure to act upon and move to effect auditory function. In particular, low mass is an important attribute of an ossicular prosthesis because it correlates directly to the force that is required for the tympanic membrane to overcome the inertia of the device and create compression waves at the oval window. Also, the recoverable strain of the shape memory polymer is orders of magnitude higher than nickel titanium shape memory alloy.

In addition, some otologists are concerned about the potential for toxicity and patient sensitivity related to the diffusion of nickel ions from nickel titanium alloy prosthesis. In distinction from nickel titanium alloy prostheses, the shape memory polymer prostheses do not include a concentration of nickel that can elute therefrom into the surrounding tissues.

Moreover, it is recognized that the use of any metal ossicular prosthesis can result in issues with magnetic resonance imaging (MRI) compatibility. MRI is an increasingly useful diagnostic tool. However, the use of MRI can induce movement of a prosthesis. If the prosthesis is moved excessively it can be displaced causing injury and/or loss of efficacy. In order to visualize the delicate structure of the ear and the surrounding brain increasingly more powerful MRI scanners are being used. Enhanced MRI compatibility is an important attribute for an ossicular prosthesis. The shape memory polymer prostheses according the invention are not affected by MRI.

By way of example, and not by limitation, the following embodiments of otological prostheses fabricated at least in part from a shape memory polymer are now described.

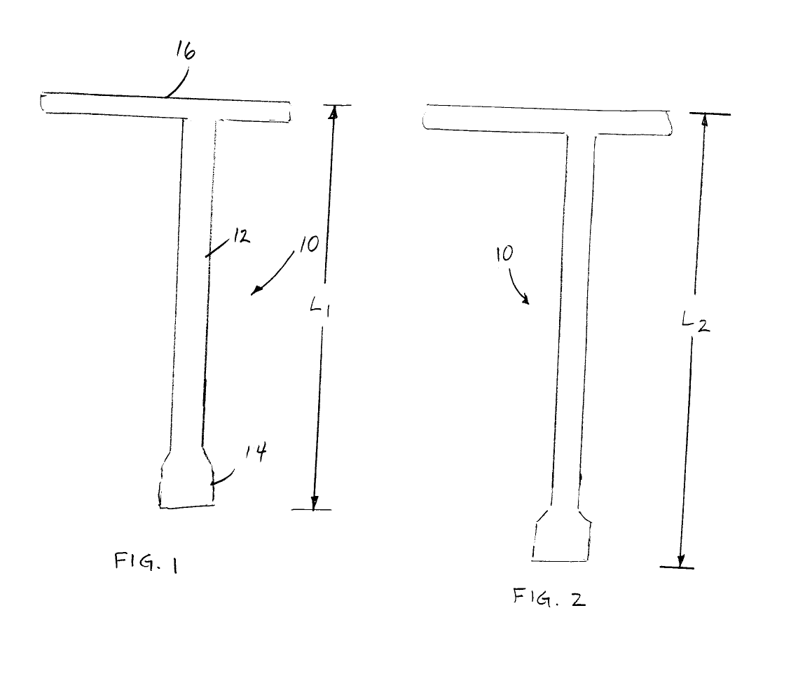

Referring to FIG. 1, a total ossicular replacement prosthesis (TORP) 10 replaces the maleus, incus and stapes. The TORP includes a shaft 12 having at an inferior end thereof a cylinder 14 for contacting the oval window and at a superior end thereof a disc 16 for contacting a tympanic membrane (ear drum). An exemplar TORP 10 is described in detail in U.S. Pat. No. 6,168,625 to Prescott, which is hereby incorporated by reference herein in its entirety. In accord with the invention, the TORP is fabricated from a shape memory polymer. The fabricated length is longer than the traditional length of a TORP, e.g., 3.0-7.0 mm. The TORP is then heated above the glass transition temperature (Tg), e.g., 75° C., and a load is applied such that the shaft 12 compresses to a length L₁ that is shorter than a (or no longer than the shortest) traditional TORP. The TORP is then cooled below Tg; i.e., quenched.

Referring to FIG. 2, prior to positioning the TORP 10 (or other ossicular prosthesis) in situ, the surgeon will determine a distance between two otological structures and then position between such structures a prosthesis having a length shorter than the measured distance. The surgeon then applies an appropriate stimulus, such as heat, to effect shape change to the TORP. Generally, within approximately 20 to 40 seconds, the device lengthens to L₂ until it makes contact with the two otologic structure (oval window, capitulum or footplate of the stapes, tympanic membrane, incus, malleus) with a predetermined contact pressure, preferably in the range of 1 to 5 MPa. The prosthesis implant will fit tight, but not too tight, so as to cause no discomfort to the recipient. The use of single size prosthesis which expands in length to accommodate all patients permits the maintenance of a reduced inventory.

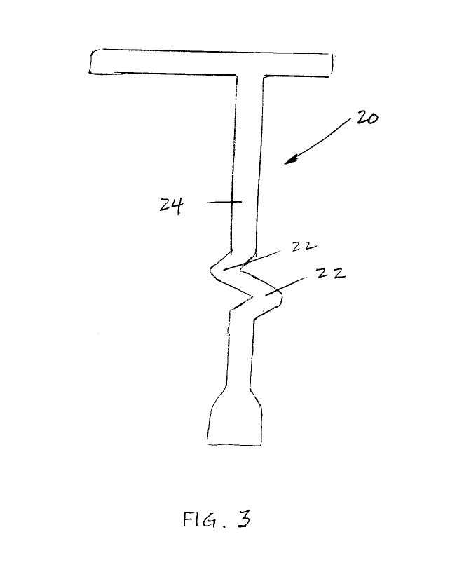

Referring to FIG. 3, it is also appreciated that length adjustment of a TORP 20 can also be accomplished by designing one or more bends 22 into the shaft 24 of the TORP and compressing the TORP about the bends 22 during heating at or above the glass transition temperature (Tg). The bend(s) may be angular or curved. The prosthesis is then quenched. Similarly, a helical coil (also shown by 22) can also be fabricated into the shaft. Then, when heated at a predetermined temperature upon implantation, e.g., 50° C., the shaft changes shape at the feature (bend, coil, twist, etc.) to effect lengthening of the shaft and contact with the appropriate anatomy.

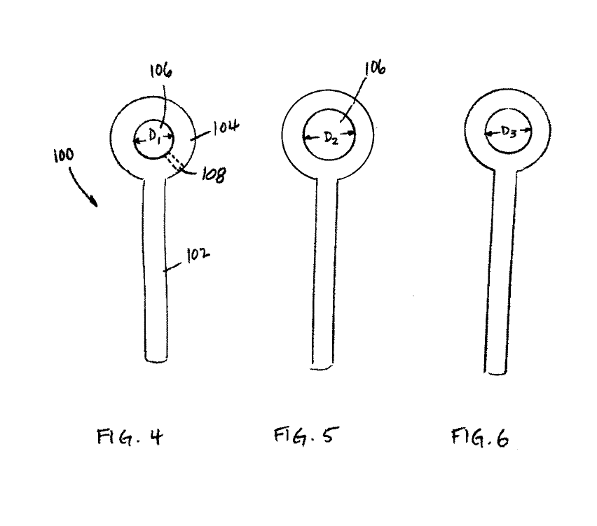

A partial ossicular replacement prosthesis, or PORP, replaces a subset of the ossicular bones (one or more, but not all). A shape memory polymer can be used for fabrication of a PORP. Turning now to FIGS. 4 through 6, by way of example, a Causse-type piston prosthesis 100 for replacement of the stapes is shown. The prosthesis 100 includes a shaft 102 and a circular head 104. The head 104 is machined with a hole 106 having a first diameter D₁, such that the hole is uninterrupted about its circumference. The hole may optionally be interrupted with a radial slot 108 shown in dotted lines. After fabrication, the prosthesis is heated above the glass transition (Tg) temperature of the shape memory polymer, e.g., 75° C., and a mandrel having an outer diameter D₂ is inserted into the hole. The prosthesis is then quenched and the mandrel is removed to maintain the diameter D₂ of the hole 106. Then, during implantation, when the surgeon applies a heat stimulus to the prosthesis at, e.g., 50° C., the inner diameter D₃ of the hole decreases to an intermediate diameter between D₂ and D₁ as it surrounds and engages a portion of the incus and couples thereto. The force of capture of the implant is less than a shape memory alloy bight and thereby prevents recipient discomfort and/or pressure induced necrosis of the bone.

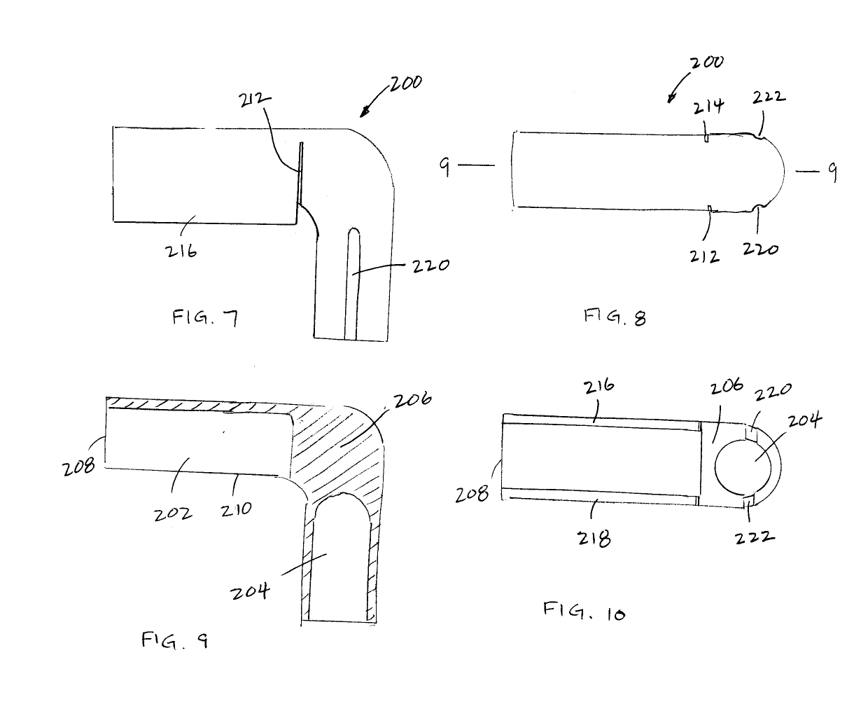

Turning now to FIGS. 7 through 10, a shape memory polymer incudo-stapedial joint (ISJ) prosthesis 200 obviates both a large inventory of different sized products as well as a complex product. The prosthesis 200 includes an incus slot 202 for receiving the long crus of the incus, and a tubular portion 204 for receiving the capitulum of the stapes. The slot 202 and tubular portion 204 are substantially transversely arranged at joint 206 so as to form a generally L-shaped structure.

More particularly, the incus slot 202 is defined from a tube 208 having a longitudinal slit 210 along an inner portion thereof (a lower surface) and opposing lateral intersecting slits 212, 214 adjacent the joint 206. Such slits 210, 212, 214 enable flaps 216, 218 of the tube 208 to be folded open to form the open slot 202. In manufacture, the flaps 216, 218 are folded open after the prosthesis is heated above the glass transition temperature and then once opened quenched to maintain such configuration until again heated above a predetermined temperature which activates the material during surgery.

The tubular portion 204 includes longitudinal slots 220, 222 for receiving the arches of the stapes through the tubular portion at any vertical location. The inner diameter of the tubular portion 204 is heated above the glass transition temperature and expanded with a mandrel to expand its size to accept practically any capitulum and then quenched to maintain such configuration until again heated above a predetermined temperature which activates the polymer during surgery.

In use, the surgeon determines the appropriate length of each of the incus slot 202 and stapedial tubular portion 204, depending the vertical and horizontal displacement of the incus and stapes. Both the slot 202 and tubular portion 204 are deep enough to accommodate vertical ‘Y’-height differences between the capitulum of the stapes and the shaft of the incus and for horizontal ‘X’-length differences. The additional polymer material, if any, at each of the portions 202, 204 can be cut and trimmed to custom fit the prosthesis to the bones. The ISJ prosthesis is then positioned to receive the capitulum with the tubular portion 204 with the arch of the stapes extending out of the slots 220, 222, and the long crus of the incus extending within the slot 202. Heat, e.g., 50° C., is then applied to the prosthesis causing the shape memory polymer to activate fixation. That is, the stapedial tubular portion 204 shrinks to secure the capitulum and the flaps 216, 218 close about the incus (re-forming a tube) to capture the incus. Only an appropriate length of each ossicle is clamped, and only with appropriate force, to prevent occluding the blood supply to the bones, and to prevent significantly limiting the bone motion and the transference of motion necessary for hearing.

There have been described and illustrated herein several embodiments of shape memory polymer otological prostheses. While particular embodiments of the invention have been described, it is not intended that the invention be limited thereto, as it is intended that the invention be as broad in scope as the art will allow and that the specification be read likewise. Thus, while TORPs, PORPs and ISJ prostheses have been generally described with reference to exemplar embodiments, it is appreciated that the invention is not limited thereto and is applicable to any suitable design of a TORP, PORP, and ISJ prosthesis, and even to any otological prosthesis. Moreover, while the exemplar embodiments are solely fabricated from a shape memory polymer, it is also appreciated that only a portion of an otological prosthesis may be fabricated from a shape memory polymer, and that other portions thereof may be fabricated from a conventional material including a metal, metal alloy, ceramic, or non-shape memory polymer. This permits the advantage of the beneficial characteristics of the various materials for different portions of the prosthesis when a non-uniform construct is desired. Also, while heat has been described in detail as the activating stimulus, it is appreciated that an activating stimulus that does not significantly alter the temperature of the polymer material can also be used, given an appropriate polymer. For example, light at an appropriate wavelength may be used to activate shape change in a stable biocompatible polymer for otological implants. It will therefore be appreciated by those skilled in the art that yet other modifications could be made to the provided invention without deviating from its spirit and scope as claimed.

What is claimed is:

1. An otological prosthesis for use between first and second otologic structures in the middle ear, comprising
a first means for engaging the first otological structure;
a second means for engaging the second otological structure; and
an intermediate portion coupling said first and second means together,
wherein at least one said first means, said second means, and said intermediate portion is made from a non-biodegradable shape memory polymer, and during manufacture is heated above a glass transition temperature of said shape memory polymer, modified in configuration, and then quenched to maintain such modified configuration until activated by a stimulus to cause the shape memory polymer to automatically assume a configuration different than said modified configuration.

2. An otological prosthesis according to claim 1, wherein:
said stimulus is heat that heats said shape memory polymer above a predetermined temperature.

3. An otological prosthesis according to claim 2, wherein:
said intermediate portion is a shaft, and said shaft changes length when heated by said heat stimulus above said predetermined temperature.

4. An otological prosthesis according to claim 3, wherein:
said shaft has a length and includes a bend along said length at which said shaft changes in length.

5. An otological prosthesis according to claim 3, wherein:
said shaft has a length and includes a coil along said length at which said shaft changes in length.

6. An otological prosthesis according to claim 3, wherein:
said first means includes a head enlarged relative to said intermediate portion, said head is provided with a hole having an inner diameter adapted to permit said head to be positioned about the first otologic structure, and when heat by said heat stimulus, said inner diameter of said head decreases such that said head is adapted to engage the first otologic structure.

7. An otological prosthesis according to claim 6, wherein:
said head has a circumference that is uninterrupted about said hole.

8. An otological prosthesis according to claim 6, wherein:
said head includes a radial slot extending into said hole.

9. An otological prosthesis according to claim 1, wherein:
said first means includes a first tubular structure comprised of said shape memory polymer, and said first tubular structure includes a longitudinal slot and defining two flaps open about said longitudinal slot that provide access into the first tubular structure along a length of said first tubular structure, the first otological structure is the incus, and the incus is able to be received into said first tubular structure through said longitudinal slot between said flaps,
wherein upon application of said stimulus, said flaps are reconfigured relative to said slot to enclose a received incus.

10. An otological prosthesis according to claim 9, wherein:
the second otological structure is the stapes, and said second means includes a tubular structure of said shape memory polymer for receiving a portion of the stapes.

11. An otologic prosthesis according to claim 10, wherein:
said second tubular structure has a diameter, and upon application of said heat stimulus, said second tubular structure is adapted to decrease in diameter.

12. An otological prosthesis according to claim 11, wherein:
said second tubular structure includes diametric slots at an end thereof for receiving opposing portions of an arch of the stapes.

13. An otological prosthesis according to claim 10, wherein:
said intermediate portion is a joint, and said first means is oriented transverse to said second means at said joint so as to form a substantially L-shaped construct with said longitudinal slot located along an inside potion of said first tubular structure.

14. An otological prosthesis according to claim 13, wherein:
an entirety of said prosthesis is constructed of said shape memory polymer.

15. An otological prosthesis according to claim 1, wherein:
said shape memory polymer is an acrylic of methacrylate.

16. An otological prosthesis according to claim 1, wherein:
said shape memory polymer is synthesized by photopolymerization from a tert-Butyl acrylate monomer with a diethyleneglycol-dimethacrylate crosslinker.

17. A method of implanting an otological prosthesis, comprising:
a) determining a distance between two otological structures;
b) delivering a prosthesis having a longest dimension shorter than the distance to a position between the two otological structures; and
c) applying a stimulus to said prosthesis causing said prosthesis to elongate and engage the two otological structures.

18. A method according to claim 17, wherein:
said prosthesis comprises a shape memory polymer.

19. A method according to claim 17, wherein:
said stimulus is heat.

20. A method according to claim 17, wherein:
said prosthesis includes an elongate shaft

21. A method of implanting an incudo-stapedial joint prosthesis, comprising:
a) providing a prostheses made of a shape memory polymer, the prosthesis including first and second tubular portions extending transversely relative to each other, said first tubular portion including a longitudinal slot about which said first tubular portion is maintained in an open configuration;
b) positioning the first tubular portion over a long crus of an incus, with the incus moved through the longitudinal slot into the first tubular portion;
c) positioning the second tubular portion over a capitulum of a stapes; and
d) applying a stimulus to the first tubular portion to close the first tubular portion relative to said longitudinal slot to capture the incus.

22. A method according to claim 21, wherein:
said second tubular portion includes two diametrical slots in an end opposite said first tubular portion, and said positioning said second tubular portion includes positioning said diametric slots over an arch of the stapes.

23. A method according to claim 21, further comprising:
applying a stimulus to the second tubular portion to cause the diameter of the second tubular portion to decrease and fixate relative to the stapes.

24. A method according to claim 21, further comprising:
trimming the length of at least one of the first and second tubular portions prior to applying to respective otological structures.
